SEQUENCE LISTING (18 sequences)

SEQ ID NO: 1 - DNA, 14 bp, artificial sequence
  catalytic core of 8-17 deoxyribozyme
tccgagccggacga

SEQ ID NO: 2 - DNA, 47 bp, artificial sequence
  IGF-S1-817
taatacgactcactatagggcgaatagccgatgtgtgagaagacctt

SEQ ID NO: 3 - DNA, 47 bp, artificial sequence
  IGF-S2-817
aaggtcttctcacacatcggctattcgccctatagtgagtcgtatta

SEQ ID NO: 4 - DNA, 47 bp, artificial sequence
  IGF-AS1-817
taatacgactcactatagggcgaataggtcttctcacacatcggctt

SEQ ID NO: 5 - DNA, 47 bp, artificial sequence
  IGF-AS2-817
aagccgatgtgtgagaagacctattcgccctatagtgagtcgtatta

SEQ ID NO: 6 - DNA, 47 bp, artificial sequence
  IGF-S1MUT-817
taatacgactcactatagggcgaatagccgatgtcacagaagacctt

SEQ ID NO: 7 - DNA, 47 bp, artificial sequence
  IGF-S2MUT-817
aaggtcttctgtgacatcggctattcgccctatagtgagtcgtatta

SEQ ID NO: 8 - DNA, 47 bp, artificial sequence
  IGF-AS1MUT-817
taatacgactcactatagggcgaataggtcttctgtgacatcggctt

SEQ ID NO: 9 - DNA, 47 bp, artificial sequence
  IGF-AS2MUT-817
aagccgatgtcacagaagacctattcgccctatagtgagtcgtatta

SEQ ID NO: 10 - DNA, 29 bp, artificial sequence
  IGF-817-8S
acatcggtccgagccggacgaattcgccc

SEQ ID NO: 11 - DNA, 29 bp, artificial sequence
  IGF-817-8-8AS
agaagactccgagccggacgaattcgccc

SEQ ID NO: 12 - DNA, 22 bp, artificial sequence
  catalytic core of 8-17 deoxyribozyme, 3′ substrate binding arm includes complement of T7 leader.
tccgagccggacgaattcgccc

SEQ ID NO: 13 - DNA, 22 bp, artificial sequence
  catalytic core of 8-17 deoxyribozyme, 3′ substrate binding arm includes complement of T7/T3 leader.
tccgagccggacgaattctccc

SEQ ID NO: 14 - DNA, 22 bp, artificial sequence
  catalytic core of 8-17 deoxyribozyme, 3′ substrate binding arm includes complement of SP6 leader.
tccgagccggacgaatgtattc

SEQ ID NO: 15 - DNA, 23 bp, artificial sequence
  Bacteriophage promoter and consensus leader sequences.
taatacgactcactatagggcga

SEQ ID NO: 16 - DNA, 23 bp, artificial sequence
  Bacteriophage promoter and consensus leader sequences.
taatacgactcactatagggaga

SEQ ID NO: 17 - DNA, 23 bp, artificial sequence
  Bacteriophage promoter and consensus leader sequences.
aattaaccctcactaaagggaga

SEQ ID NO: 18 - DNA, 23 bp, artificial sequence
  Bacteriophage promoter and consensus leader sequences.
atttaggtgacactatagaatac

(19) United States
(12) Patent Application Publication
(10) Pub. No.: US 2004/0086860 A1
(43) Pub. Date: May 6, 2004

(54) Methods of producing RNAs of defined length and sequence

(76) Inventor: Muhammad Sohail, Marston (GB)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/264,748

(22) Filed: Oct. 4, 2002

(51) Int. Cl. C12Q 1/68; C12P 19/34

(52) U.S. Cl. 435/6; 435/91.2

(57) ABSTRACT

Methods of making RNA duplexes and single-stranded RNAs of a desired length and sequence based on cleavage of RNA molecules at a defined position, most preferably with the use of deoxyribozymes. Novel deoxyribozymes capable of cleaving RNAs including a leader sequence at a site 3′ to the leader sequence are also described.

### FIELD OF THE INVENTION

The invention relate to methods of making RNA duplexes and single-stranded RNAs of a desired length and sequence based on cleavage of RNA molecules at a defined position, most preferably with the use of deoxyribozymes.

### BACKGROUND TO THE INVENTION

Small interfering RNAs (siRNAs) are powerful laboratory tools for directed post-transcriptional gene expression knockdown (Elbashir et al., 2001, Lewis et al., 2002; Harborth et al., 2001) and inhibition of viral propagation (Jacque et al., 2002; Gitlin et al, 2002; Jiang and Milner, 2002). The mechanism of action of siRNAs remains largely elusive. Data to-date suggest that siRNAs may bind to the target mRNA and serve as primers for an RNA-dependent RNA polymerase to convert it into dsRNA. An RNase III-type enzyme cleaves dsRNA to produce a pool of 21-23 nt or 24-26 nt long dsRNA fragments, thus amplifying the effect of original siRNA. The cellular machinery then uses this new set of siRNAs to repeat the process, silencing expression of the target gene (Lipardi et al., 2001; Zamore et al., 2000; Ketting et al., 2001; Elbashir et al., 2001; Hamilton A et al., 2002). Specific enzymes involved in RNAi are largely unknown. However, an evolutionarily conserved family of cellular RNase III proteins (named Dicer enzymes) containing an ATP-dependent helicase-type domain, two RNase III-type domains and a dsRNA-binding motif appears to be at the heart of RNAi (Filippov et al., 2000; Elbashir et al., 2001; Bernstein et al., 2001; Hannon, 2002). Genetic studies in Drosophila, Neurospora, Arabidopsis and C. elegans have also revealed several other candidate genes for potential roles in RNAi. (Williams and Rubin, 2002; Sharp P A, 2001; Tuschl, 2001; Hannon, 2002).

Exogenous siRNA is frequently used in RNAi studies. Using chemically synthesised RNA oligonucleotides, Elbashir et al. (2001) described a systematic analysis of the optimal Drosophila siRNA duplex. Based on their suggestions, exogenous siRNAs typically consist of a double-stranded region of 19 base pairs and two nucleotides 3′ overhangs: -TT-3′/-UU-3′ overhangs are preferred over other sequences. For siRNA to be active, it is important that the overhang in the antisense strand is complementary to the target messenger RNA. Probably, due to secondary structure in mRNAs, siRNA targeted to different regions of a gene are not equally potent in inhibiting gene expression (Holen et al., 2002; Zhou et al., 2002). Therefore, it may be desirable to screen several siRNAs to obtain reagents with optimal activity.

There are three general methods of producing RNA fragments: chemical synthesis, intra cellular expression and in vitro transcription. These methods have their advantages and disadvantages depending on the application.

Chemical synthesis of RNAs is relatively straightforward but is expensive. Furthermore, it is difficult to synthesise chemically RNA fragments that are longer than ˜50 nts.

Intracellular expression requires cloning of a DNA fragment into an expression vector, usually under the control of an inducible promoter. Although, this method provides a source of continuous production of RNA in the cell, it offers little control over the quantity of the expressed RNA and the sequence length.

In vitro transcription is relatively cheap and offers a good approach to synthesis of large quantities of RNA. Commonly a DNA-dependent RNA polymerase of bacteriophage origin is used for in vitro transcription. These RNA polymerases require a specific promoter sequence for binding on the template DNA and, for optimal activity, require a downstream sequence called the “leader sequence”. The leader sequence appears at the 5′-end of the in vitro transcripts and may be unsuitable in several applications, such as in siRNA-mediated RNA interference.

Donze and Picard (2002) and Yu et al. (2002) both describe an in vitro transcription method for production of siRNAs, which is based on the use of oligonucleotides as templates to produce short transcripts (as first described by Milligan and Uhlenbeck (1989)). This method is relatively simple and cheap but is limited by specific sequence requirements: all siRNAs made with this method start with a 5′-G residue and require a C-3′ residue at position 19 (i.e., 5′-G-N17-C-3′) to allow annealing with the complementary RNA which also has to start with a 5′-G residue due to the requirement by the T7 RNA polymerase. Since it is important that the overhang in the antisense strand is complementary to the target mRNA, therefore, if the optimal TT-3′/UU-3′ overhang is used in siRNA, the mRNA sequence needs to be 5′-AAG (i.e., mRNA is 5′-AAG-N17-C-3′). Efficacy of siRNAs targeted to different regions of a gene varies dramatically. Therefore, these strict sequence requirements greatly reduce the number of potential target sites for siRNA selection and are thus disadvantageous in the identification of optimally effective siRNAs. A further disadvantage with this method is that it is not possible to use a leader sequence in conjunction with the T7 promoter, since the leader sequence would be transcribed and incorporated into the siRNA and would ultimately prevent the siRNA from functioning in RNA interference.

The present invention seeks to provide improved methods of production of RNAs of defined length and sequence, particularly siRNAs, by incorporation of an RNA cleavage step in order to remove unwanted sequences.

### SUMMARY OF THE INVENTION

In a first aspect the invention provides a method of producing an RNA duplex having a defined length and sequence, the method comprising:

providing a first primary single-stranded RNA and cleaving the first primary RNA at a defined position to generate a first RNA strand having a defined length and sequence,

providing a second RNA strand having a defined length and sequence, wherein the first and second RNA strands are of complementary sequence over at least a portion of their length, and

annealing the first and second RNA strands to form an RNA duplex.

The invention further provides a method of producing an RNA strand having a defined length and sequence, comprising:

producing a primary single-stranded RNA including a cleavage site, wherein the RNA sequence upstream of the cleavage site comprises a leader sequence, and the RNA sequence downstream of the cleavage site consists of the defined RNA sequence required in the RNA strand, and

cleaving the primary RNA at the cleavage site, thereby generating an RNA strand having the required length and sequence.

The invention still further provides a deoxyribozyme or ribozyme comprising a 5′ substrate binding arm, a catalytic core and a 3′ substrate binding arm, wherein the 3′ substrate binding arm is capable of specifically hybridising to a leader sequence present in an RNA molecule under conditions of high stringency.

### BRIEF DESCRIPTION OF THE DRAWINGS

FIG. 1 is a flowchart summarising an example of a procedure for siRNA synthesis according to the invention. The 3′ arm of the deoxyribozyme is complementary to the bacteriophage promoter sequence and the 5′ arm to the target mRNA sequence. Two complementary oligonucleotides are annealed to produce template for RNA polymerase. Each strand of the siRNA is prepared in a separate in vitro transcription reaction. The transcripts are digested with an appropriate deoxyribozyme to remove extra ribonucleotides. The two complementary RNA strands are annealed to obtain siRNA. This example is intended to be illustrative of rather than limited to the invention.

FIG. 2 illustrates use of siRNAs specific for human IGF1R mRNA produced according to the invention in an RNAi experiment. (A): Production of single-stranded RNAs with deoxyribozyme digestion. The transcripts were labelled with ³³P. In controls (SM and ASM), the central three nucleotides were modified (see Table 2). It was therefore possible to use only two deoxyribozymes to digest all four RNA fragments. (B): Activity of the siRNAs in cell cultures against the target mRNA (IGF1R); 22-chemically synthesised siRNA, 22inv-inverted control, SM/ASM-modified variant control, S/AS-enzymatically synthesised IGF1R-specific siRNA (C): Quantitation of the inhibition.

### DETAILED DESCRIPTION OF THE INVENTION

Methods of Producing RNA

The invention provides methods of making RNA duplexes and single-stranded RNAs of a desired length and sequence, based on cleavage of RNA molecules at a defined position.

The term “RNA duplex” encompasses RNA molecules that are double-stranded over at least a portion of their length. The double-stranded region may be flanked at the 5′ and/or 3′ end by single-stranded overhangs. The term “RNA duplex” also encompasses RNA hairpin structures (such as the hairpin siRNAs described by Yu et al. 2002)

The methods of the invention are most preferred for use in the synthesis of small interfering RNAs (siRNAs), which are a particular class of RNA duplex. SiRNAs are characterised by their short length, typically comprising or consisting of a double-stranded region of less than 30 base-pairs, more preferably from 19 to 22 base-pairs, and most preferably 19 base-pairs. This double-stranded region may be flanked by short single-stranded overhangs. Typically, siRNAs include short (several nucleotide) overhangs at the 3′ end, with the 3′ overhang in the antisense strand being complementary to the target mRNA strand. It is most preferred to include 3′ dinucleotide overhangs, the most preferred being -UU-3′. SiRNAs may also be formed as hairpin RNAs, in which both strands of the siRNA duplex are included within a single RNA strand (Yu, et al. 2002).

The methods of the invention rely on cleavage of primary single-stranded RNAs at a defined position in order to generate RNA strands of a required length and sequence. If it is desired to produce an RNA duplex then two such RNA strands having complementary sequence, over at least a portion of their length, may be synthesised and then annealed to form an RNA duplex.

Cleavage of the primary RNA strands at a defined position (the cleavage site) is most preferably carried out with the use of a deoxyribozyme (also referred to as DNA enzymes or DNAzymes). Deoxyribozymes are catalytic DNA molecules that can cleave RNA, typically at a site that contains a specific di-nucleotide catalytic sequence. Several general classes of deoxyribozymes capable of cleaving RNA strands at different di-nucleotide catalytic sites are known in the art (Santoro and Joyce, 1997; Feldman and Sen, 2001, the contents of which are incorporated herein by reference).

Ribozymes (catalytic RNA molecules) of the required specificity may be substituted for deoxyribozymes in the methods of the invention with equivalent effect, but it is preferred to use deoxyribozymes because they are easier and cheaper to produce than ribozymes and less sensitive to chemical and enzymatic degradation. Use of deoxyribozymes provides an additional advantage that it is possible to use digestion with DNase in order to separate the deoxyribozyme from the desired RNA cleavage products, as illustrated in the examples.

Ribozymes are generally known in the art, and the sequence/structural requirements in order to produce a ribozymes capable of cleaving an RNA strand in trans at a defined cleavage site are well characterised. Most preferably the ribozyme will be a hammerhead ribozyme (Pley, H. W. et al. Nature. 372: 68-74 (1994); Scott, W. G. et al. Cell. 81: 991-1002 (1995)).

The primary RNAs, which are cleaved to generate RNA strands of the required length and sequence, must include the desired RNA sequence (the RNA sequence which is required in the final RNA product, which may be referred to herein as the “downstream sequence” or “target-specific sequence”) positioned immediately downstream of the cleavage site. The sequence upstream of the cleavage site, referred to herein as the “upstream sequence”, will be removed by the cleavage reaction and is not incorporated into the final RNA product; hence its precise sequence is not material to the invention. However, in the preferred embodiment, wherein the cleavage reaction is carried out with the use of a deoxyribozyme (or ribozyme), the “upstream sequence” must contain a sequence capable of hybridising with the enzyme such that the catalytic core of the enzyme is correctly positioned to cleave the RNA strand at the defined cleavage site.

The “upstream sequence” may also be selected to provide some desirable property in order to'enhance the overall performance of the method. For example, the upstream sequence may be selected to improve the efficiency of the in vitro transcription reaction and/or to enhance the stability of the primary RNA transcript (prior to cleavage). In a preferred embodiment the “upstream sequence” may comprise a “leader sequence”. A “leader sequence” is a sequence which, when positioned immediately downstream of an RNA polymerase-dependent promoter, becomes incorporated into the 5′ ends of RNA transcripts initiating at the promoter, and functions to enhance efficiency of the transcription reaction.

The most preferred leader sequences for use in the invention are the consensus leader sequences associated with the bacteriophage T7, T3 and SP6 promoters. These sequences are listed in Table 1, below.

The T7, T3 and SP6 consensus leader sequences are short (only 6 nt in length) and it is difficult to achieve specific hybridisation of a deoxyribozyme (or ribozyme) to such a short sequence. It is therefore within the scope of the invention to incorporate one or more additional spacer nucleotides between the leader sequence and the chosen cleavage site in order to provide a longer upstream sequence for deoxyribozyme binding. The number of additional spacer nucleotides and their precise sequence is not material to the invention and may be selected by the user as appropriate. The class of deoxyribozyme which it is desired to use in the cleavage step, and in particular minimal length requirements of the binding arm of the deoxyribozyme which will bind to the upstream sequence, are key factors in determining the minimum length of the upstream sequence, since the upstream sequence must be at least long enough to hybridise with this binding arm. The length of the chosen leader sequence will determine the number of additional nucleotides required in order to provide a binding site of adequate length in the upstream sequence. By way of example, when using T7, T3 or SP6 leader sequences of 6 nt in length with 8-17 deoxyribozymes it is generally sufficient to add a further 2 spacer nucleotides between the leader sequence and the dinucleotide forming the cleavage site. These two spacer nucleotides may be of any sequence, provided that they base-pair with corresponding nucleotides in the deoxyribozyme.

The precise sequence of the “cleavage site”, this being the point at which the starting RNA strand is cleaved to generate a downstream RNA strand of defined length and sequence, will vary depending on the nature of the deoxyribozyme. Various classes of deoxyribozymes (and ribozymes) are known in the art that are capable of cleaving target RNAs at different di-nucleotide catalytic sequences.

Deoxyribozymes consist of a catalytic domain flanked by two substrate-specific 7-8nt binding arms. The target RNA is cleaved between two specific nucleotides (Santoro and Joyce, 1997; Feldman and Sen, 2001). The requirement for this di-nucleotide sequence is different for different deoxyribozyme groups making them a flexible tool for digesting a variety of sequences. For example, 10-23 type deoxyribozymes (Santoro and Joyce, 1997) can digest an RNA strand containing the sequences 5′AT, 5′AC, 5′GC and 5′GT; 8-17 type deoxyribozymes (Santoro and Joyce, 1997) cleave at 5′AG and Bipartite II deoxyribozymes (Feldman and Sen, 2001) optimally cleave at 5′AA. The underlined nucleotides are unpaired. In the catalytic sites for 10-23 and 8-17 deoxyribozymes the second nucleotides pair with a complementary nucleotide in the deoxyribozyme. In optimally active Bipartite II deoxyribozymes the binding arms are separated by five unpaired nucleotides, including those at the catalytic site (Feldman and Sen, 2001). Therefore, any combination of upstream and downstream sequences can be cleaved at a defined di-nucleotide cleavage (catalytic) site with the use of an appropriate deoxyribozyme.

Deoxyribozymes, or ribozymes, for use in the methods of the invention may be conveniently prepared by chemical synthesis using standard oligonucleotide synthesis techniques. Design and synthesis of appropriate deoxyribozymes will be further understood with reference to the description given below and the accompanying Examples.

When the primary RNA is cleaved at a specific di-nucleotide using a deoxyribozyme, the 3′-most residue of this di-nucleotide forms the 5′ end of the downstream RNA strand. If this RNA strand is to be incorporated into an siRNA, then it may be important for the performance of the siRNA in RNA interference that this nucleotide matches up with the target sequence for the siRNA in the target gene.

The primary single-stranded RNAs are most preferably synthesised by in vitro transcription, a standard molecular biology technique well known to those skilled in the art (see Sambrook et al. (1989), Molecular cloning: A Laboratory Manual, Cold Spring Harbor Laboratory Press). The only requirement of the in vitro transcription reaction is that it should generate an RNA transcript which includes the desired RNA sequence positioned downstream of a cleavage site. Hence, in vitro transcription may be carried out using any suitable technique known in the art.

In a preferred embodiment in vitro transcription may be carried out using the method of Milligan and Uhlenbeck (1989) (the contents of which are incorporated herein by reference), which is based on the use of oligonucleotides to produce short transcripts. A similar approach has been applied in the production of siRNAs by Donze and Picard (2002) and Yu et al. (2002), the contents of which are incorporated herein by reference.

For convenience it is generally preferred to use bacteriophage RNA polymerases, e.g. T7, T3 or SP6 polymerase for the in vitro transcription reaction because they are readily available and produce large quantities of transcript.

When the method of the invention is used to synthesise an RNA duplex, such as an siRNA, it is preferred to make the two RNA strands separately, most preferably by independent in vitro transcription and cleavage reactions, and then anneal the two strands to form the RNA duplex. It is also contemplated to synthesise both the first and second RNA strands in a single primary RNA, which is then cleaved internally at a second cleavage site in order to separate the first and second RNA strands.

In a further embodiment of the invention the RNA duplex may be a hairpin RNA, wherein both strands of the RNA duplex are included within a single RNA molecule which is self-complementary over at least a portion of its length. In vitro-transcribed hairpin siRNAs are described by Yu et al. (2002). For synthesis of a hairpin RNA according to the invention a single RNA strand is synthesised by in vitro transcription, then cleaved to remove unwanted sequences from the 5′ end. The resulting RNA strand is then self-annealed to from the hairpin structure.

Therefore, in a further aspect the invention provides a method of producing a hairpin RNA duplex having a defined length and sequence comprising:

providing a primary single-stranded RNA and cleaving the RNA at a defined position to generate an RNA of defined length and sequence which is self-complementary over at least a portion of its length, and self-annealing the RNA to form a hairpin RNA duplex.

In the most preferred embodiments of the methods of the invention, wherein the primary RNAs are synthesised by in vitro transcription (most preferably driven by a DNA oligonucleotide) and a deoxyribozyme is used to cleave the primary RNAS, the method of the invention provides a considerable cost saving over chemical synthesis in the production of RNA duplexes, even taking into account the need to provide DNA oligonucleotides for use as the deoxyribozymes and possibly DNA oligonucleotide templates for the in vitro transcription reaction. Chemical synthesis of DNA oligonucleotides is considerably less expensive than synthesis of RNA oligonucleotides.

The methods of the invention are particularly suited to the production of large-scale quantities of RNA (e.g. large scale synthesis of siRNAs for use as pharmaceutical agents) or to the production of libraries of RNA molecules of varying length and sequence (e.g. libraries of slightly varying siRNAs required for optimisation of RNAi to any given target gene), which might otherwise be prohibitively expensive were it necessary to rely on chemical synthesis of RNA.

The inclusion of a cleavage step to remove unwanted sequences from the 5′ end of a primary RNA transcript has particular advantages in the production of siRNAs, where the precise sequence of the ends of the siRNA duplex are important in allowing the siRNA to interact with the target gene through RNA interference. As aforesaid, previous methods of producing siRNAs by in vitro transcription using T7 polymerase have inherent sequence constraints, because efficient T7 polymerase initiation requires the first nucleotide of each RNA to be G. This strict requirement greatly reduces the number of potential target sites for siRNA-mediated RNA interference in any given gene. The present invention avoids this limitation by cleavage of the RNA at a defined position to remove nucleotides at the extreme 5′ end of the RNA.

The inclusion of the cleavage step also enables the user to include sequences in the 5′ end of the primary transcript, e.g. leader sequences, in order to increase the efficiency of in vitro transcription. Having served its intended purpose of enhancing in vitro transcription the leader sequence is cleaved off and thus does not appear in the final RNA product. In other words, the ability to selectively remove unwanted regions from the 5′ end of a primary transcript by a highly specific and controlled cleavage reaction (e.g. using a deoxyribozyme) allows the user to maximise the efficiency of the in vitro transcription reaction used to produce the primary RNA without compromising the function of the final (i.e. cleaved) RNA product.

The technical advantages provided by the method are not limited to the production of siRNAs, but extend to production single-stranded RNAs of defined length and sequence. In particular, the advantage of improved transcription efficiency by incorporating of a leader sequence also applies to the production of single-stranded RNAs. Single-stranded RNAs having a defined length and sequence may be useful as antisense reagents, or as research tools, for example in structural studies or for the study of interactions between RNA and other molecules such as proteins, other nucleic acids and drugs.

Novel Deoxyribozymes and Deoxyribozyme Precursors

In a further aspect the invention relates to deoxyribozymes (and ribozymes) specifically designed for cleavage of RNA transcripts incorporating a leader 5′ sequence.

In particular, the invention provides a deoxyribozyme (or ribozyme) comprising a 5′ substrate binding arm, a catalytic core and a 3′ substrate binding arm, wherein the 3′ substrate binding arm is capable of specifically hybridising to a region of a target RNA molecule including a leader sequence, thereby enabling the deoxyribozyme to cleave the target RNA molecule at a site 3′ of the leader sequence.

The phrase “capable of specifically hybridising” may be taken to mean that when included in a deoxyribozyme the 3′ substrate binding arm is capable of hybridising to a region of the target RNA under the reaction conditions generally used for cleavage of target RNAs with deoxyribozymes (e.g. 50 mM Tris-HCl pH 7.5, 10 mM MgCl₂, 150 mM NaCl at 37° C.). Such conditions would be well known to those skilled in the art. “Specific hybridization” of the 3′ binding arm of the deoxyribozyme to a particular region of the target RNA should be taken to mean that when incorporated into a deoxyribozyme the 3′ binding arm can form a stable duplex with a complementary region in the target RNA including a “leader sequence” under the conditions used for RNA cleavage with the deoxyribozyme, and that under these conditions the 3′ binding arm does not to any significant extent form a duplex with other regions of the target RNA.

The general features of deoxyribozymes are well known in the art (Santoro and Joyce, 1997; Feldman and Sen, 2001). Deoxyribozymes generally comprise a catalytic core, which includes the residues required for cleavage of the target RNA strand, flanked by 5′ and 3′ substrate binding arms which bind to complementary sequences in the target RNA and correctly position the catalytic core for cleavage of the target RNA. The deoxyribozyme provided by the invention may belong to the 8-17, 10-23 or bipartite II classes of deoxyribozyme, the general features of these classes of deoxyribozymes being known in the art (Santoro and Joyce, 1997 for 8-17 and 10-23; Feldman and Sen, 2001 for bipartite II). However, other types of deoxyribozymes having the desired function of cleaving a target RNA downstream of a leader sequence are also contemplated.

The deoxyribozyme (or ribozyme) of the invention functions to cleave a target RNA downstream of (i.e. 3′ to) a leader sequence. In this context the term “leader sequence” refers to a feature which is present in RNA. The leader sequence is typically a bacteriophage consensus leader sequence, associated with a bacteriophage promoter/RNA polymerase system. The preferred leader sequences are those associated with the T7 promoter (5′-gggcga-, or 5′-gggaga-), T3 promoter (5′-gggaga-), or SP6 promoter (5′-gaauac-), since these promoters (and their corresponding polymerases) are most commonly used in production of RNAs by in vitro transcription.

In a preferred embodiment the 3′ substrate binding arm of the deoxyribozyme includes a sequence which is complementary to the leader sequence, this complementary sequence most preferably sharing 100% sequence identity with the complement of the leader sequence. However, sequences sharing less than 100% identity with the complement of the leader sequence may still allow the 3′ substrate binding arm to bind to the leader sequence in the target RNA with sufficiently high binding affinity to enable correct positioning of the deoxyribozyme for cleavage of the target RNA. Such sequences are therefore not excluded.

In a preferred embodiment the 3′ substrate binding arm of the deoxyribozyme may comprise a sequence selected from the group consisting of: -tcgccc-3′ (complement of T7 consensus leader), -tctccc-3′ (complement of T3 and T7 consensus leaders), and -gtattc-3′ (complement of SP6 consensus leader).

The region of complementarity between the 3′ substrate binding arm of the deoxyribozyme and the target RNA may extend beyond the leader sequence if the leader sequence is short. There is generally a minimal length requirement for the 3′ as well as 5′ substrate binding arms, which is determined by the need for the deoxyribozyme to bind specifically to its target RNA. The length of the 3′ substrate binding arm of the deoxyribozyme may vary depending on the class of deoxyribozyme, but will typically be at least 7 nt. The optimum length of the 3′ binding arm will vary depending on the target sequence, and the precise reaction conditions under which it is intended to use the deoxyribozyme (Werner, M. & Uhlenbeck, O. C. (1995) Nucleic Acids Res. 23: 2092-2096; Hegg, L. A. & Fedor, M. J. (1995) Biochemistry. 34: 15813-15828; Hertel, K. J. et al. (1996) EMBO J. 15: 3751-3757). Hence, for any given target RNA the skilled reader will readily be able to design a deoxyribozyme containing binding arms of optimal length by variation of these factors.

The sequence of the “catalytic core” of the deoxyribozyme will vary depending on the chosen class of deoxyribozyme, i.e. 8-17, 10-23 or bipartite II deoxyribozyme, this in turn being dependent on the sequence of the target RNA which it is desired to cleave using the enzyme. As aforesaid, the different classes of deoxyribozymes cleave target RNA molecules at different di-nucleotide catalytic sites. The minimal catalytic motifs for each of these classes of deoxyribozyme have been well characterised (see Santoro and Joyce, 1997 for 8-17 and 10-23 ribozymes; Feldman and Sen, 2001 for bipartite II ribozymes).

Preferred, non-limiting, examples of deoxyribozymes provided by the invention are those having a sequence selected from the group consisting of:

5′ R¹—R²-tcgccc

5′ R¹—R²-tctccc

5′ R¹—R²-gtattc

5′ R¹—R²-n_{(N)}-tcgccc

5′ R¹—R²-n_{(N)}-tctccc

5′ R¹—R²-n_{(N)}-gtattc

wherein R¹ represents a 5′ substrate binding arm sequence, R² represents a deoxyribozyme catalytic core sequence, n represents a,t,c or g, and N is a positive integer, greater than or equal to 1.

The notation -n_{(N)}- represents spacer nucleotides inserted in order to lengthen the 3′ substrate binding arm. The precise number and sequence of these spacer nucleotides will vary depending on the sequence surrounding the leader sequence in the target RNA, and on the precise location of the site at which it is desired to cleave the target RNA, relative to the leader sequence. However, N will typically be 5 or less, most preferably 1, 2 or 3.

The catalytic core sequence represented as “R²” is most preferably a catalytic core sequence of an 8-17, 10-23 or bipartite II deoxyribozyme.

The most preferred catalytic core sequence for 8-17 deoxyribozymes is: tccgagccggacga (SEQ ID NO:1).

As will be apparent to the skilled reader, the precise length and sequence of the 5′ substrate binding arm (represented as “R¹”) will vary depending on the nature of the target RNA which it is desired to cleave using the deoxyribozyme. The 5′ substrate binding arm will preferably, but need not necessarily, be 100% complementary to the target RNA. It may be possible for the deoxyribozyme to function effectively will a lesser degree of complementarity between the 5′ binding arm and the substrate, for example if the 5′ binding arm is long.

Preferred, non-limiting examples of 8-17 deoxyribozymes according to the invention are as follows:

5′ R¹-tccgagccggacga-attcgccc (8-17 catalytic core, 3′ substrate binding arm includes complement of T7 leader),

5′ R¹-tccgagccggacga-attctccc (8-17 catalytic core, 3′ substrate binding arm includes complement of T7/T3 leader) and

5′ R¹-tccgagccggacga-atgtattc (8-17 catalytic core, 3′ substrate binding arm includes complement of SP6 leader)

wherein R¹— represents the 5′ substrate binding arm of the deoxyribozyme.

The deoxyribozymes will most preferably be single-stranded DNA molecules, but may incorporate modified (i.e. non-natural) back-bone linkages and/or modified bases, provided that these modifications do not inhibit the function of the deoxyribozyme in cleavage of the target RNA to a material extent. The deoxyribozymes may be chemically synthesised using standard apparatus and techniques for oligonucleotide synthesis.

Chemical synthesis of oligonucleotides is generally carried out on a solid support, such as controlled pore glass (CPG) or polystyrene. In routine oligonucleotide synthesis, bases are added one-by-one to the growing chain in a 3′ to 5′ direction (opposite to enzymatic synthesis by DNA polymerases). It is therefore contemplated to prepare “universal” deoxyribozyme precursor structures, comprising the 3′ substrate binding arm and catalytic core of a deoxyribozyme according to the invention attached to a solid support at the 3′ end and having a free 5′ end for the addition of further bases. These “part-complete” precursor structures could be supplied to an end-user attached to a suitable solid support compatible with the end-user's DNA synthesis apparatus. The end-user may then simply extend the DNA chain by the addition of further bases to form the desired 5′ substrate binding arm.

The catalytic core of the deoxyribozyme is constant for a given class of deoxyribozymes and the 3′ substrate binding arm can be designed for removal of leader sequences associated with use of a particular in vitro transcription system. For any given in vitro transcription system the leader sequence can remain constant, regardless of the sequence of the remainder of the transcript.

A set of “universal” deoxyribozyme precursors may be readily synthesised to match the most commonly used in vitro transcription systems, i.e. those based on T7, T3 or SP6 polymerases, and their corresponding promoter and consensus leader sequences.

Preferred, non-limiting, examples of deoxyribozyme precursor structures according to the invention include the following:

5′ R²-tcgccc-X

5′ R²-tctccc-X

5′ R²-gtattc-X

5′ R²-n_{(N)}-tcgccc-X

5′ R²-n_{(N)}-tctccc-X 5′ R²-n_{(N)}-gtattc-X

wherein R² represents a deoxyribozyme catalytic core sequence, —X represents a linkage to a solid support, n represents a,t,c or g, and N is a positive integer, greater than or equal to 1.

The catalytic core sequence represented as “R²” is most preferably a catalytic core sequence of an 8-17, 10-23 or bipartite II deoxyribozyme.

The most preferred catalytic core sequence for 8-17 deoxyribozymes is: tccgagccggacga (SEQ ID NO:1)

Preferred non-limiting examples of 8-17 deoxyribozyme precursor structures according to the invention include the following:

5′ tccgagccggacgaattcgccc-X (8-17 catalytic core, 3′ binding arm includes complement of T7 leader sequence),

5′ tccgagccggacgaattctccc-X, (8-17 catalytic core, 3′ binding arm includes complement of T7/T3 leader sequence), and

5′ tccgagccggacgaatgtattc-X (8-17 catalytic core, 3′ binding arm includes complement of SP6 leader sequence)

wherein —X represents a linkage to a solid support.

The “solid support” may be essentially any type of solid support suitable for use in chemical synthesis of DNA oligonucleotides and may vary depending upon the method/apparatus which it is intended to use for DNA synthesis.

Examples of suitable solid supports include controlled pore glass (CPG) and polystyrene.

The skilled reader will appreciate that it is possible to produce ribozymes (catalytic RNA molecules) having equivalent specificity to the deoxyribozymes of the invention using techniques known in the art for the production of ribozymes. Hence, the invention also extends to ribozymes capable of cleaving a target RNA at a site 3′ to a leader sequence present in the target RNA.

Ribozymes are generally known in the art, and the sequence/structural requirements in order to produce a ribozymes capable of cleaving an RNA strand in trans at a defined cleavage site are well characterised. Most preferably the ribozyme will be a hammerhead ribozyme (Pley, H. W. et al. Nature. 372: 68-74 (1994); Scott, W. G. et al. Cell. 81: 991-1002 (1995)). Hammerhead ribozymes include two stems or helices which base-pair with complementary sequences in the target RNA; Stem III (or helix III) is analogous to the 3′ substrate binding arm of the deoxyribozyme and base-pairs with the target RNA upstream of (5′ to) the cleavage site, whereas Stem I (or helix I) is analogous to the 5′ substrate binding arm of the deoxyribozyme and base-pairs with the target RNA upstream of (3′ to) the cleavage site. Hence, in hammerhead ribozymes according to the invention it is stem I of the ribozyme which should be capable of specifically hybridising with the leader sequence in the target RNA. References in the claims to “3′ substrate binding arm” and “5′ substrate binding arm” can therefore be construed as referring to stem III and stem I, respectively, according to the standard nomenclature for hammerhead ribozymes.

The invention will be further understood with reference to the following experimental examples:

### EXAMPLE 1—

### Production of siRNAs Specific for Human IGF1R mRNA

siRNAs specific for the human insulin-like growth factor receptor (IGF1R) mRNA were produced using T7 RNA polymerase generated transcripts and 8-17 type deoxyribozymes.

A structurally accessible region of the IGF1R mRNA was selected for targeting with siRNAs. The template oligonucleotides for producing siRNAs were designed such that the resultant siRNA would have characteristics proposed by Elbashir et al. (2001).

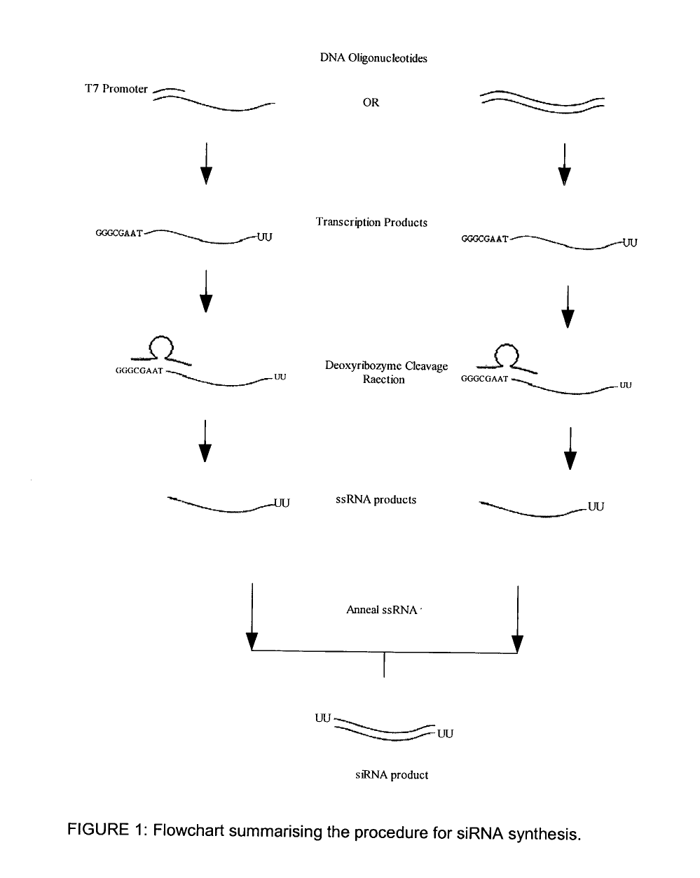

The two strands of the siRNA were produced in separate in vitro transcription reactions. Two complementary oligonucleotides were designed to provide templates for RNA polymerase. The oligonucleotides included sequence for binding T7 RNA polymerase (other RNA polymerases could be substituted with equivalent effect), a T7 leader sequence and an IGF1R-specific sequence. The commonly used hexa-nucleotide leader sequence of the T7 promoter, GGGAGA or GGGCGA (Table 1) appears in the transcripts. Therefore, one arm of the deoxyribozyme (3′ arm) can be designed to bind to this leader sequence in RNA and the other (5′ arm) to the target gene sequence cassette (in this example IGF1R). However, 8-17 deoxyribozymes have been shown to work optimally with 7-8nt binding arms (Santoro and Joyce, 1997). Therefore, two additional spacer nucleotides were included downstream of the T7 leader sequence to provide a longer annealing site for the 3′ arm of the deoxyribozyme. 5′ AT was chosen to extend the promoter sequence, since catalytic sites surrounded by A or U appear to be a better substrate for the deoxyribozyme (Santoro and Joyce, 1997); 5′T (of 5′AT extension) also helped to trace the 5′ cleavage product with the use of either [α-³²P]UTP or [a-³³P]UTP in in vitro transcription reactions. A third nucleotide, 5′A, was added that, with the first nucleotide of the target (IGF1R) sequence (5′G in this case), made the catalytic site for the deoxyribozyme (5′AG). 5′TT/3′AA was added after the target gene sequence to provide a 5′UU overhang in the siRNA. Two 21-mer complementary RNA transcripts were synthesised using this approach and annealed to produce siRNA with a 19 base-pair double-stranded region and 3′ UU overhangs. A flow chart summarising the production of siRNAs with this method is given in FIG. 1.

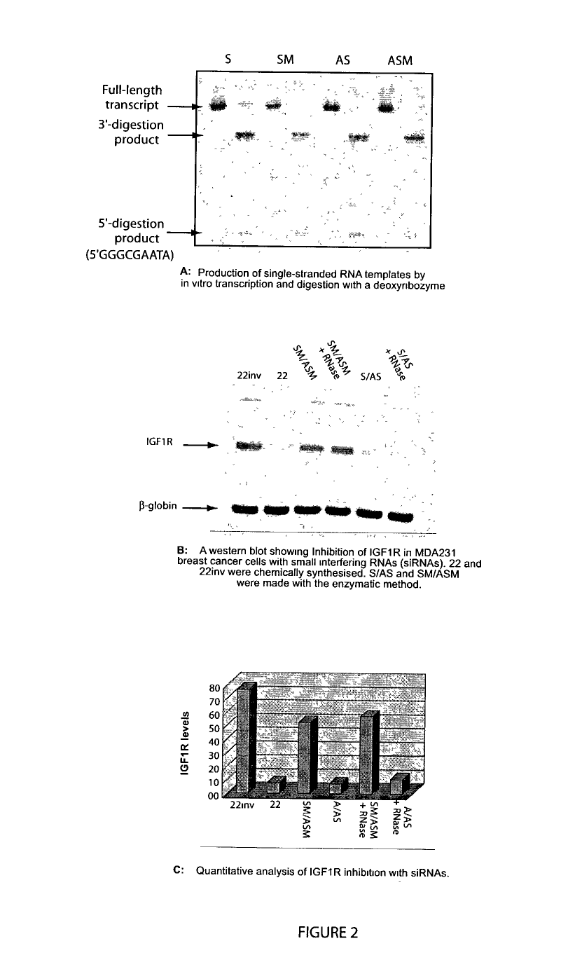

The annealed RNAs (siRNAs) were tested for their ability to inhibit IGF1R production in MDA231 breast cancer cells. The activity of siRNAs produced by the enzymatic method (referred to herein as S/AS) was compared with chemically synthesised siRNA (referred to herein as “22”); both had identical sequences. Negative controls were “22inv” (inverted 22 sequence, chemically synthesised) and “SM/ASM” (with three nt mismatch, produced enzymatically). Results are shown in FIG. 2. At 50 nM concentration, both 22 and S/AS inhibited IGF1R production to similar extent. The SM/ASM variant also inhibited expression of IGF1R, but the extent of inhibition was approximately 50% less than that observed with 22 and S/AS. Overall, the results suggest that the method described herein produced siRNA of comparable quality with commercial chemically synthesised siRNA.

Materials and Methods

Oligonucleotides and Purification

Oligonucleotides were synthesised on an ABI394 DNA/RNA synthesizer using standard nucleotide CE-phosphoramidites (Cruachem) and were desalted through NAP-5 columns (Amersham). Purity of the oligonucleotides was evaluated on a 12% denaturing polyacrylamide gel after end-labelling with ³²P in the presence of [γ-³²P]ATP (>5000 Ci/mmol; Amersham) and T4 polynucleotide kinase (Roche Diagnostics). Oligonucleotide stocks were maintained at 100 pmol/μl concentration in water at −20° C. A list of oligonucleotides and deoxyribozymes is provided in Table 2. If required, oligonucleotides may be purified, for example by HPLC or gel electrophoresis or any other suitable purification technique known in the art.

Table 1: Bacteriophage promoter and consensus leader sequences (in italics). The leader sequences appear in transcripts

TABLE 2
Oligonucleotide and deoxyribozyme sequences

| | |
|---|---|
| T7 RNA | 5′ taa tac gac tca cta ta ggg cga |
| Polymerase | |
| T7 RNA | 5′ taa tac gac tca cta ta ggg aga |
| polymerase | |
| T3 RNA | 5′ aat taa ccc tca cta aa ggg aga |
| polymerase | |
| SP6 RNA | 5′ att tag gtg aca cta ta gaa tac |
| polymerase | |

| | |
|---|---|
| Name | Sequence |

Template oligonucleotides to make sense (S) strand of siRNA

| | |
|---|---|
| IGF-S1-817 | 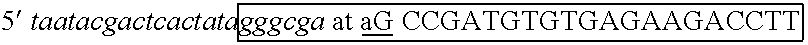 |
| IGF-S2-817 | 5′AAGGTCTTCTCACACATCGG Ct at tcgccctatagtgagtcgtatta |

Template oligonucleotides to make anti-sense (AS) strand of siRNA

| | |
|---|---|
| IGF-AS1-817 | 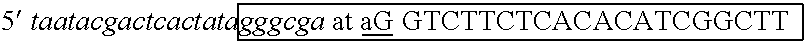 |
| IGF-AS2-817 | 5′AAGCCGATGTGTGAGAAGAC Ct at tcgccctatagtgagtcgtatta |

Template oligonucleotides to make mutant sense (SM) strand of siRNA

| | |
|---|---|
| IGF-S1MUT-817 | 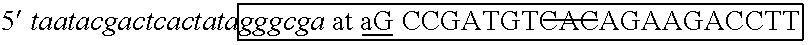 |
| IGF-S2MUT-817 | 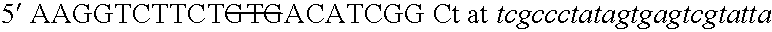 |

Template oligonucleotides to make mutant anti-sense (ASM) strand of siRNA

| | |
|---|---|
| IGF-AS1MUT-817 | 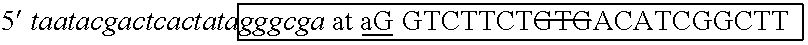 |
| IGF-AS2MUT-817 | 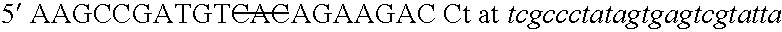 |

Deoxyribozyme to digest S and SMUT strands of siRNA

| | |
|---|---|
| IGF-817-8-8S | 5′aca tcg gTc cga gcc gga cga att cgc cc |

Deoxyribozyme to digest AS and ASMUT strands of siRNA

| | |
|---|---|
| IGF-817-8-8AS | 5′aga aga cTc cga gcc gga cga att cgc cc |

GUIDE for oligonucleotides:
Italics: T7 promoter sequence. [symbol]
T7 promoter sequence added to transcripts.
Bold: Additional nucleotides added between promoter and the target sequence/catalytic
site to increase length for deoxyribozyme binding.
Underlined: Catalytic site for 8-17 deoxyribozymes
CAPITALS: IGF1R sequence
CAPITALS/Bold: 3′ 2nt overhang

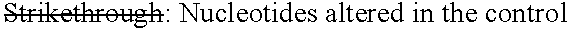

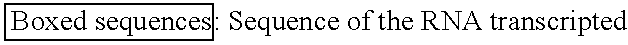

GUIDE for deoxyribozymes:
Bold: Binding arms.
Underlined: Constant sequence of the doexyribozyme: 5′Twobble-pairs with 3′G in the
target sequence.
Italics: Catalytic domain of 8-17 deoxyribozymes.

In Vitro Transcription

In vitro transcription reactions were carried out at 37° C. for 4 hr using MegaScript or MegaShortScript kits (Ambion) mainly according to the manufacturer's supplied protocol. Two complementary oligonucleotides were annealed to produce template for the transcription reactions. 100 pmol of the annealed template was used in a 20 μl reaction containing 225 nmol of each NTP and 3-4 μl of the T7 enzyme mix supplied with the Ambion kits. In labelling reactions, 1 μl of either [α-³²P]UTP (3000Ci/mmol; Amersham) or [-³³P]UTP (2500Ci/mmol; Amersham) was added as tracer to assess the quality of transcripts. After transcription, 1U of RNase-free DNase (Ambion) was added to digest the template (37° C. for 20 min) to avoid interfere with the subsequent deoxyribozyme digestion. The reaction was diluted to 37 μl with water, passed through a Sephadex G25 Microspin column (Amersham) and eluted into a microfuge tube containing 2 μl of 10x deoxyribozyme reaction buffer (see below) and 20U of RNasin (Promega).

Deoxyribozyme Reactions

In in vitro transcription reactions using T7/T3 or SP6 promoters/RNA polymerases, a 5′ leader sequence is added to the transcripts. This needs to be removed to obtain single-stranded RNA templates for siRNA of desired sequence and length. Deoxyribozymes were used to remove unwanted nucleotides from transcripts. Typically, a deoxyribozyme reaction was carried out in 20 μl volume containing approximately 100-120 μM transcripts and 100 μM deoxyribozyme in 100 mM NaCl, 30 mM MgCl2, 20 mM Tris-HCl pH 7.2 and 20U of RNasin (Promega). The reaction was incubated overnight at RT or in a thermal cycler (MJ Research) (cycling at 50° C. for 1 min, 20° C. for 2 min and 30° C. 10 min). Finally, DNase was added to digest deoxyribozyme that could interfere with the annealing of the siRNA strands by competing with complementary sequences. The reaction was purified through a Sephadex G25 Microspin column (Amersham). Sense and antisense strands were prepared in separate reactions and were annealed (at 70° C. for 2 min and cooled to room temperature) to obtain siRNA duplexes. SiRNA duplexes were maintained at ˜50 μM concentration in 0.5×deoxyribozyme reaction buffer at −20° C.

If required, siRNAs may be purified, for example by HPLC or gel electrophoresis or any other suitable purification technique known in the art.

Processing of Chemically Synthesised siRNAs

RNA/DNA chimeric oligonucleotides were designed as described (ElBashir et al. 2001). The sequence was the same as that for enzymatically prepared RNAs (Table-2), except that each strand had 19 bases of RNA with two 3′ deoxythymidine nucleotides. A duplex with inverted sequence was used as control. The oligonucleotides were HLPC purified (Transgenomic Laboratories, Glasgow UK) and complementary strands were annealed (at 85° C. for 1 min and at 37° C. for 1 hr) in 100 mM CH₃COOK, 30 mM Hepes-KOH pH 7.4, 2 mM (CH₃COO)₂Mg. Duplex formation was confirmed by electrophoresis through 5% low melting temperature agarose (NuSieve GTG, FMC BioProducts, Rockland Me.).

Cell Culture and Transfection

Human ER negative breast cancer cell line MDA-MB-231 was obtained from the Cancer Research UK Cell Production Laboratories, South Mimms, UK. The cells were cultured in RPMI-1640 medium with 10% FCS and were negative on testing for mycoplasma infection. Cells were transfected with siRNAs using Oligofectamine (InVitrogen) according to the manufacturer's instructions. After 48 hr the cells were lysed and cell extracts analysed as previously described (Macaulay et al. 2001) by immunoblotting for IGF1R (antibody supplied by Santa-Cruz) and β-tubulin (antibody supplied by Sigma).

### REFERENCES

The contents of the documents listed below are to be incorporated into the present application by reference:

Bernstein E, Caudy A A, Hammond S M, Hannon G J. Role for a bidentate ribonuclease in the initiation step of RNA interference. Nature 409, 363-366 (2001).

Donze O, Picard D. RNA interference in mammalian cells using siRNAs synthesized with T7 RNA polymerase. Nucleic Acids Res. 30, e46 (2002).

Elbashir S M, Lendeckel W, Tuschl T. RNA interference is mediated by 21- and 22-nucleotide RNAs. Genes Dev. 15, 188-200 (2001).

Elbashir S. Martinez J. Patkaniowska A. Lendeckel W. Tuschl T. Functional anatomy of siRNAs for mediating efficient RNAi im Drospophila melanogaster embryo lysate. EMBO J. 20, 6877-6888 (2001).

Feldman A R, Sen D. A new and efficient DNA enzyme for the sequence-specific cleavage of RNA. J. Mol. Biol. 313, 283-294 (2001).

Filippov V, Solovyev V, Filippova M, Gill S S. A novel type of RNase III family proteins in eukaryotes. Gene 245, 213-221 (2000).

Gitlin L, Karelsky S, Andino R. Short interfering RNA confers intracellular antiviral immunity in human cells. Nature 418, 430-434 (2002).

Hamilton A, Voinnet O, Chappell L, Baulcombe D. Two classes of short interfering RNA in RNA silencing. EMBO J. 21, 4671-4679 (2002).

Hannon G J. RNA interference. Nature 418, 244-251 (2002).

Harborth J, Elbashir S M, Bechert K, Tuschl T, Weber K. Identification of essential genes in cultured mammalian cells using small interfering RNAs. J Cell Sci. 114, 4557-4565 (2001).

Holen T, Amarzguioui M, Wiiger M T, Babaie E, Prydz H. Positional effects of short interfering RNAs targeting the human coagulation trigger Tissue Factor. Nucleic Acids Res. 30, 1757-1766 (2002).

Jacque J M, Triques K, Stevenson M. Modulation of HIV-1 replication by RNA interference. Nature 418, 435-438 (2002).

Jiang M, Milner J. Selective silencing of viral gene expression in HPV-positive human cervical carcinoma cells treated with siRNA, a primer of RNA interference. Oncogene 21, 6041-6048 (2002).

Ketting R F, Fischer S E, Bernstein E, Sijen T, Hannon G J, Plasterk R H. Dicer functions in RNA interference and in synthesis of small RNA involved in developmental timing in C. elegans. Genes Dev. 15, 2654-2659 (2001).

Lewis D L, Hagstrom J E, Loomis A G, Wolff J A, Herweijer H. Efficient delivery of siRNA for inhibition of gene expression in postnatal mice. Nat Genet. 32, 107-108 (2002)

Lipardi C, Wei Q, Paterson B M. RNAi as random degradative PCR: siRNA primers convert mRNA into dsRNAs that are degraded to generate new siRNAs. Cell 107, 297-307 (2001).

Milligan J F, Uhlenbeck O C. Synthesis of small RNAs using T7 RNA polymerase. Methods Enzymol. 180, 51-62 (1989).

Santoro S W, Joyce G F. A general purpose RNA-cleaving DNA enzyme. Proc. Natl. Acad. Sci. 94, 4262-4266 (1997).

Sharp P A RNA interference—2001. Genes Dev. 15, 485-490 (2001).

Tuschl T. RNA interference and small interfering RNAS. Chembiochem 2, 239-245 (2001).

Williams R W, Rubin G M. ARGONAUTE1 is required for efficient RNA interference in Drosophila embryos. Proc. Natl. Acad. Sci. 99, 6889-6894 (2002).

Yu et al. RNA interference by expression of short-interfering RNAs and hairpin RNAs in mammalian cells. Proc. Natl. Acad. Sci USA, Vol: 99, pp 6047-6052 (2002).

Zamore P D, Tuschl T, Sharp P A, Bartel D P. RNAi: double-stranded RNA directs the ATP-dependent cleavage of mRNA at 21 to 23 nucleotide intervals. Cell 101, 25-33 (2000).

Zhou Y. et al. Post-transcriptional suppression of gene expression in Xenopus embryos by small interfering RNA. Nucleic Acids Res. 30, 1664-1669 (2002).

1. A method of producing an RNA duplex having a defined length and sequence comprising:
providing a first primary single-stranded RNA and cleaving this RNA at a defined position to generate a first RNA strand having a defined length and sequence,
providing a second RNA strand having a defined length and sequence, wherein the first and second RNA strands are of complementary sequence over at least a portion of their length, and
annealing the first and second RNA strands to form an RNA duplex.

2. A method according to claim 1 wherein the second RNA strand is produced by cleaving a second primary single-stranded RNA at a defined position to generate an RNA strand of the required length and sequence.

3. A method according to claim 1 wherein the second RNA strand is produced by cleaving the first primary single-stranded RNA at a second defined position.

4. A method according to claim 1 wherein cleavage of RNA at a defined position is carried out using a deoxyribozyme or ribozyme.

5. A method according to claim 1 wherein the first primary RNA is synthesised by in vitro transcription.

6. A method according to claim 5 wherein the first primary RNA comprises upstream and downstream RNA sequences separated by a cleavage site, wherein the upstream RNA sequence comprises a leader sequence, and the downstream RNA sequence consists of the sequence of the first RNA strand.

7. A method according to claim 6 wherein the leader sequence is a bacteriophage promoter consensus leader sequence.

8. A method according to claim 7 wherein the leader sequence is a T7, T3 or SP6 consensus leader sequence.

9. A method according to claim 8 wherein the leader sequence comprises one of the following sequences:
5′-gggcga, 5′-gggaga, or 5′-gaauac.

10. A method according to claim 2 wherein the second primary RNA is synthesised by in vitro transcription.

11. A method according to claim 10 wherein the second primary RNA comprises upstream and downstream RNA sequences separated by a cleavage site, wherein the upstream RNA sequence comprises a leader sequence, and the downstream RNA sequence consists of the sequence of the second RNA strand

12. A method according to claim 1 wherein the RNA duplex is a small interfering RNA.

13. A method according to claim 12 wherein the small interfering RNA comprises a double-stranded region of less than 30 base pairs in length.

14. A method according to claim 13 wherein the double-stranded region is flanked by 3′ overhangs of -UU-3′.

15. A method of producing a hairpin RNA duplex having a defined length and sequence comprising:
providing a primary single-stranded RNA and cleaving the RNA at a defined position to generate an RNA of defined length and sequence which is self-complementary over at least a portion of its length, and self-annealing the RNA to form a hairpin RNA duplex.

16. A method of producing an RNA strand having a defined length and sequence, comprising:
producing a primary single-stranded RNA including a cleavage site, wherein the RNA sequence upstream of the cleavage site comprises a leader sequence, and the RNA sequence downstream of the cleavage site consists of the defined RNA sequence required in the RNA strand, and
cleaving the primary RNA at the cleavage site, thereby generating an RNA strand having the required length and sequence.

17. A method according to claim 16 wherein the leader sequence is a bacteriophage promoter consensus leader sequence.

18. A method according to claim 17 wherein the leader sequence is a T7, T3 or SP6 consensus leader sequence.

19. A method according to claim 18 wherein the leader sequence comprises one of the following sequences:
5′-gggcga, 5′-gggaga, or 5′-gaauac.

20. A deoxyribozyme or ribozyme comprising a 5′ substrate binding arm, a catalytic core and a 3′ substrate binding arm, wherein the 3′ substrate binding arm is capable of specifically hybridising to a region of a target RNA molecule including a leader sequence under conditions of high stringency thereby enabling the deoxyribozyme to cleave the target RNA molecule at a site 3′ of the leader sequence.

21. A deoxyribozyme or ribozyme according to claim 20 wherein the 3′ substrate binding arm includes a sequence which is complementary to the leader sequence.

22. A deoxyribozyme or ribozyme according to claim 20 or claim 21 wherein the leader sequence is a bacteriophage consensus leader sequence.

23. A deoxyribozyme or ribozyme according to claim 22 wherein the leader sequence is a T7, T3 or SP6 consensus leader sequence.

24. A deoxyribozyme or ribozyme according to claim 23 wherein the leader sequence is selected from the group consisting of: 5′-gggcga-, 5′-gggaga-, and 5′-gaauac-.

25. A deoxyribozyme according to claim 21 wherein the 3′ substrate binding arm includes a sequence selected from the group consisting of: -tcgccc-3′, -tctccc-3′, and -gtattc-3′.

26. A deoxyribozyme according to claim 25, having a sequence selected from the group consisting of:
5′R¹-R²-tcgccc
5′ R¹-R²-tctccc
5′ R¹-R²-gtattc
5′ R¹-R²-n_{(N)}-tcgccc
5′ R¹-R²-n_{(N)}-tctccc
5′ R¹-R²-n_{(N)}-gtattc
wherein R′ represents a 5′ substrate binding arm sequence, R² represents a deoxyribozyme catalytic core sequence, n represents a,t,c or g, and N is a positive integer, greater than or equal to 1.

27. A deoxyribozyme according to claim 26 wherein R² is a catalytic core sequence of an 8-17, 10-23 or bipartite II deoxyribozyme.

28. A deoxyribozyme according to claim 27, which is an 8-17 deoxyribozyme having a catalytic core sequence: tccgagccggacga

29. A deoxyribozyme according to claim 28, which is an 8-17 deoxyribozyme having a sequence selected from the group consisting of:
5′ R¹-tccgagccggacga-at-tcgccc
5′ R¹-tccgagccggacga-at-tctccc
5′ R¹-tccgagccggacga-at-gtattc
wherein R¹— represents a 5′ substrate binding arm sequence.

30. A deoxyribozyme precursor comprising a fragment of a deoxyribozyme including the 3′ substrate binding arm and catalytic core linked at the 3′ end to a solid support, wherein the 3′ substrate binding arm is capable of hybridising under conditions of high stringency to a leader sequence present in an RNA molecule.

31. A deoxyribozyme precursor according to claim 30 wherein the 3′ substrate binding arm includes a sequence which is complementary to the leader sequence.

32. A deoxyribozyme precursor according to claim 31 or claim 32 wherein the leader sequence is a bacteriophage consensus leader sequence.

33. A deoxyribozyme precursor according to claim 32 wherein the leader sequence is a T7, T3 or SP6 consensus leader sequence.

34. A deoxyribozyme precursor according to claim 33 wherein the leader sequence is selected from the group consisting of: 5′-gggcga-, 5′-gggaga-, and 5′-gaauac-.

35. A deoxyribozyme precursor according to claim 34 wherein the 3′ substrate binding arm includes a sequence selected from the group consisting of: -tcgccc-3′, -tctccc-3′, and -gtattc-3′.

36. A deoxyribozyme precursor according to claim 35, having one of the following structures:
5′ R²-tcgccc-X
5′ R²-tctccc-X
5′ R²-gtattc-X
5′ R²-n_{(N)}-tcgccc-X
5′ R²-n_{(N)}-tctccc-X
5′ R²-n_{(N)}-gtattc-X
wherein R² represents a deoxyribozyme catalytic core sequence, —X represents a linkage to a solid support, n represents a,t,c or g, and N is a positive integer, greater than or equal to 1.

37. A deoxyribozyme precursor according to claim 36 wherein R² is a catalytic core sequence of an 8-17, 10-23 or bipartite II deoxyribozyme.

38. A deoxyribozyme precursor according to claim 37, which is an 8-17 deoxyribozyme having the catalytic core sequence: tccgagccggacga.

39. A deoxyribozyme according to claim 38, which is an 8-17 deoxyribozyme having a structure selected from the group consisting of:
5′ tccgagccggacgaattcgccc-X
5′ tccgagccggacgaattctccc-X
5′ tccgagccggacgaatgtattc-X
wherein —X represents a linkage to a solid support.
